(19) United States
(12) Patent Application Publication
(10) Pub. No.: US 2014/0137966 A1
(43) Pub. Date: May 22, 2014

(54) VALVE MECHANISM

(71) Applicant: Michael Harms, Frankfurt am Main (DE)

(72) Inventor: Michael Harms, Frankfurt am Main (DE)

(73) Assignee: SANOFI-AVENTIS DEUTSCHLAND GMBH, Frankfurt am Main (DE)

(21) Appl. No.: 14/128,023

(22) PCT Filed: Jun. 21, 2012
(86) PCT No.: PCT/EP2012/061915

§ 371 Date: Dec. 20, 2013

(30) Foreign Application Priority Data
Jun. 22, 2011 (EP) 11170898.8

(51) Int. Cl. A61M 39/22; A61M 5/168

(52) U.S. Cl. CPC A61M 39/22; A61M 5/168
USPC 137/602

(57) ABSTRACT

The invention provides a medical device having a valve mechanism which has an effective sealing, is simple and cost-saving to produce and at the same time provides an improved pressure control. Additionally, an improved biocompatibility can be provided. The medical device comprises a channel and at least one valve element. The channel comprises at least two openings, and the valve element comprises at least one sealing area. The valve element can be brought in a closed state, such that the sealing area of the valve element seals one of the openings of the channel and the valve element is at least in part flexible, such that the valve element can be brought into an open state by the pressure of a medium inside the channel and the medium can exit the channel between the valve element and the channel.

### CROSS REFERENCE TO RELATED APPLICATIONS

The present application is a U.S. National Phase Application pursuant to 35 U.S.C. §371 of International Application No. PCT/EP2012/061915 filed Jun. 21, 2012, which claims priority to European Patent Application No. 11170898.8 filed Jun. 22, 2011. The entire disclosure contents of these applications are herewith incorporated by reference into the present application.

### TECHNICAL FIELD

The present patent application relates to medical devices for delivering at least two drug agents from separate reservoirs. Such drug agents may comprise a first and a second medicament. The medical device includes a dose setting mechanism for delivering the drug agents automatically or manually by the user.

The medical device can be an injector, for example a hand-held injector, especially a pen-type injector, that is an injector of the kind that provides for administration by injection of medicinal products from one or more multidose cartridges. In particular, the present invention relates to such injectors where a user may set the dose.

The drug agents may be contained in two or more multiple dose reservoirs, containers or packages, each containing independent (single drug compound) or pre-mixed (co-formulated multiple drug compounds) drug agents.

### BACKGROUND

Certain disease states require treatment using one or more different medicaments. Some drug compounds need to be delivered in a specific relationship with each other in order to deliver the optimum therapeutic dose. The present patent application is of particular benefit where combination therapy is desirable, but not possible in a single formulation for reasons such as, but not limited to, stability, compromised therapeutic performance and toxicology.

For example, in some cases it may be beneficial to treat a diabetic with a long acting insulin (also may be referred to as the first or primary medicament) along with a glucagon-like peptide-1 such as GLP-1 or GLP-1 analog (also may be referred to as the second drug or secondary medicament).

Accordingly, there exists a need to provide devices for the delivery of two or more medicaments in a single injection or delivery step that is simple for the user to perform without complicated physical manipulations of the drug delivery device. The proposed drug delivery device provides separate storage containers or cartridge retainers for two or more active drug agents. These active drug agents are then combined and/or delivered to the patient during a single delivery procedure. These active agents may be administered together in a combined dose or alternatively, these active agents may be combined in a sequential manner, one after the other.

### SUMMARY

The drug delivery device also allows for the opportunity of varying the quantity of the medicaments. For example, one fluid quantity can be varied by changing the properties of the injection device (e.g., setting a user variable dose or changing the device's “fixed” dose). The second medicament quantity can be changed by manufacturing a variety of secondary drug containing packages with each variant containing a different volume and/or concentration of the second active agent.

The drug delivery device may have a single dispense interface. This interface may be configured for fluid communication with a primary reservoir and with a secondary reservoir of medicament containing at least one drug agent. The drug dispense interface can be a type of outlet that allows the two or more medicaments to exit the system and be delivered to the patient.

The combination of compounds from separate reservoirs can be delivered to the body via a double-ended needle assembly. This provides a combination drug injection system that, from a user's perspective, achieves drug delivery in a manner that closely matches the currently available injection devices that use standard needle assemblies. One possible delivery procedure may involve the following steps:

1. Attach a dispense interface to a distal end of the electro-mechanical injection device. The dispense interface comprises a first and a second proximal needle. The first and second needles pierce a first reservoir containing a primary compound and a second reservoir containing a secondary compound, respectively.

2. Attach a dose dispenser, such as a double-ended needle assembly, to a distal end of the dispense interface. In this manner, a proximal end of the needle assembly is in fluidic communication with both the primary compound and secondary compound.

3. Dial up/set a desired dose of the primary compound from the injection device, for example, via a graphical user interface (GUI).

4. After the user sets the dose of the primary compound, the micro-processor controlled control unit may determine or compute a dose of the secondary compound and preferably may determine or compute this second dose based on a previously stored therapeutic dose profile. It is this computed combination of medicaments that will then be injected by the user. The therapeutic dose profile may be user selectable. Alternatively, the user can dial or set a desired dose of the secondary compound.

5. Optionally, after the second dose has been set, the device may be placed in an armed condition. The optional armed condition may be achieved by pressing and/or holding an “OK” or an “Arm” button on a control panel. The armed condition may be provided for a predefined period of time during which the device can be used to dispense the combined dose.

6. Then, the user will insert or apply the distal end of the dose dispenser (e.g. a double ended needle assembly) into the desired injection site. The dose of the combination of the primary compound and the secondary compound (and potentially a third medicament) is administered by activating an injection user interface (e.g. an injection button).

Both medicaments may be delivered via one injection needle or dose dispenser and in one injection step. This offers a convenient benefit to the user in terms of reduced user steps compared to administering two separate injections.

The medicaments in above mentioned devices are generally guided by fluidic channels with diameters similar to or slightly bigger than common injection needles. It is especially challenging to provide an effective seal with such small dimensions, which can be provided at a low cost.

In the state of the art, valves, such as umbrella valves, are often used. They usually consist of elastic materials like TPE (thermoplastic elastomeres). It is disadvantageous though to use such materials, since they normally contain plasticisers or softening agents, in order to provide an effective seal, which can be opened and closed multiple times with a reasonable force. These chemicals are often not biocompatible and can cause various side effects for the user, because the chemicals can diffuse into the guided fluids.

Moreover, in case at least two medicaments are delivered by the medical device, these medicaments often need to be delivered successively. This can be necessary to avoid contaminations of one medicament with the other medicament or to reduce or better control the pressure, with which the medicaments are ejected from the medical device into the skin of the user.

Thus the invention faces the technical problem of providing a valve mechanism for a medical device which valve provides an effective sealing, is simple and cost-saving to produce and at the same time provides an improved pressure control. Additionally an improved biocompatibility should be provided.

The technical problem is solved by a medical device comprising a channel comprising at least two openings, a valve element comprising at least one sealing area. The valve element is at least in part flexible and is configured to have a closed state, in which the sealing area of the valve element seals one of the openings of the channel, wherein the valve element is further configured to have an open state such that said valve element can be brought into the open state by the pressure of a medium inside the channel, and the medium can exit the channel between the valve element and the channel and wherein said valve element (300, 300′) is at least partially made of metal.

By providing a valve element, which is at least in part flexible the medium inside the channel can bring the valve element into an open state. For this to happen the force originating from pressure inside the medium needs to be sufficiently high, so that it is higher than the force by the valve element acting against the pressure of the medium inside the channel. With an at least in parts flexible valve element, complicated mechanisms can be avoided and a simple and cost-saving production can be achieved. Moreover such a simple mechanism is less prone to errors. Since the force, with which the sealing area of the valve element seals the opening, can be adjusted by choosing the material or designing the flexibility of the valve element, for example by varying the thickness or varying the shape of the valve element, not only an effective sealing can be provided but also the pressure of the medium exiting the valve element can be controlled.

The term “open state” is understood to mean that there must not necessarily be a single open state. Since the pressure and thus the force on the valve element can grow continuously there might be multiple open states for the valve element with possibly different cross sections between valve element and channel for the medium to go through. It might as well be possible that an open state or any open state can only be achieved if a certain threshold in the pressure is exceeded.

The medium exits the channel in between the valve element and the channel. In this way, it is possible to dispense with chemicals like softening agents and make use of different materials. Umbrella valves, for example, need to be very flexible so that the medium can exit through the middle of the umbrella valve, which had to be realized with unwanted chemicals. The use of materials like metal would not be possible in such a case. Additionally a valve element like an umbrella valve needs to be tightly attached to the channel, which again necessitates the use of chemicals like adhesives. Now that the medium exits between the valve element and the channel, other materials, like metal, can be utilized and a higher degree of biocompatibility of the valve element can be achieved. Moreover the valve element does not need to be fixed to the channel with adhesives or the like, since the medium is supposed to exit the channel between channel and valve element and not through the valve element.

In particular, the channel can be a needle or a cannula. Such a needle can be connected to a drug reservoir on the one side, while the other opening can be sealed by the valve element.

Multiple of such valve elements can be provided in a medical device. For example, there can be provided a y-channel, which first guides two medicaments separately by the first and second arm of the y-channel and which then guides them through the common third arm of the y-channel to an injection site. A valve element according to the invention can be implemented before or in the two first arms of the y-channel, making it possible to guide the two medicaments through the common third arm successively by increasing the pressure in each channel successively. That means that first the pressure in the first medicament is increased, such that the first valve element is brought into an open state and after the pressure in the first medicament is decreased again and the first valve element is in a closed state, the pressure in the second medicament can be increased, such that the second valve element is brought into an open state. Finally the pressure in the second medicament is decreased again, and the second valve element is brought in a closed state again. As a result, the first medicament is flowing when the second medicament is not and vice versa. Of course, this also works with other mediums and an arbitrary number of channels or valve elements.

As can be seen, the use of a medical device according to the invention is especially useful, since an effective sealing, a cost-saving production, an improved pressure control and an improved biocompatibility are desirable features for medical devices.

According to a second embodiment of the invention, the sealing area of the valve element, when in the closed state, seals the opening of the channel by direct contact with the channel. In this way, no further elements like membranes need to be used. This further facilitates the production process and nevertheless provides an effective sealing. Moreover the risk of introducing non-biocompatible substances is reduced by using fewer elements made of different materials.

In a further embodiment of the invention, the sealing area of the valve element can be bent by the pressure of the medium inside the channel in the axial direction of the channel and away from the channel. In this way, the force acting against the valve element can be efficiently used by pressing or bending away the sealing area of the valve element in the axial direction of the channel. In this way, the opening between the channel and the valve element is maximized with a low force, providing in particular an improved pressure control.

The valve element is at least partially made of metal, in particular titanium or steel. Moreover, the whole valve element can be made of metal, in particular inert metals like titanium or steel. Especially steels like V2A or V4A are applicable. Since these metals are substantially inert with respect to chemical reactions with mediums used in the medical area, the biocompatibility is significantly improved, especially in comparison with valves made of TPE. Additionally the production can be facilitated by using above mentioned materials.

It is likewise preferred that the channel is at least in part made of metal, in particular titanium or steel. Moreover the whole channel can be made of metal, in particular inert metals like titanium or steel, for example V2A or V4A steels. Again, the production can be facilitated by using these materials.

In an example embodiment, the valve element is substantially flat. Substantially flat means that its width and length are significantly larger than its height. In this way, a space saving implementation of a valve can be achieved. Moreover a practicable way of controlling the ejection force or pressure after the valve element is attained by adjusting the height of the valve element and thus controlling its flexibility or deformability.

According to another embodiment of the invention the valve element, in its closed state, is curved towards the channel. The sealing of the opening is improved by a valve element with this shape, since due to a curved valve element its stability is enhanced.

In a preferred embodiment, the valve element is pre-stressed, such that the valve element returns into the closed state if the pressure in the medium drops below a threshold. The pre-stressed state can be achieved by forming the valve element, in particular a valve element made of metal, in such a way, that a spring-like force acts in the direction of the opening, as it is done with leaf springs, for example. On the one hand the sealing is made safer in this way, since the valve element presses against the opening, and on the other hand the valve element closes automatically, when the pressure in the medium drops below a threshold. This threshold is defined by the point, where the force due to the pressure of the medium drops below the counteracting force of the pre-stressed valve element. A pre-stress can also account for faster closing times, since the valve element, does not need to be closed manually or by an external impulse.

In a preferred embodiment, the valve element shows hysteresis during its opening and closing. That means that the relation between force or pressure on the valve element and its displacement is not only dependent on the current force but also on the internal state of the valve element or on the way the valve element came into the current state. By providing a valve element like this, the threshold for opening the valve element can be higher than the threshold for closing the valve element, for example. If there is no hysteresis, these thresholds are substantially the same.

Since the pressure in the medium may drop as soon as the valve element is brought in an open state, it is especially advantageous to provide a valve element, which shows hysteresis to prevent the valve element 300 from an uncontrolled opening and closing movement.

Such a hysteresis can be implemented either by the choice of the material of the valve element or by a treatment of the valve element like forming operations providing a pre-stress, for example.

In an example embodiment, the valve element has a meta-stable open state. A meta-stable state is understood as a state of the valve element, into which the valve element can be brought and where only a small force is needed to bring the valve element out of said state. Such a meta-stable state could be provided by a metal plate, which can flip back and forth between a convex and a concave state, for example. Depending on the pre-stress, the valve element either remains in said meta-stable open state, even if the pressure of the medium inside the channel drops again, for example to ambient pressure, or the valve element is automatically brought out of the meta-stable state into the closed state again. In either way the meta-stable state provides a better controlled open state, which is less dependent on the pressure of the medium.

If the valve element is configured such that it remains in the open state, even if the pressure in said medium drops, pressure variations do not change the degree to which the valve is opened. This can be achieved, if the pre-stress is low enough, for example. With a significant hysteresis the force or pressure necessary to open the valve element can be designed higher than the force or pressure necessary to keep the valve element open. In case a meta-stable open state is provided, the valve element can also be brought back into the closed state by an external mechanical impulse, for example.

In a further embodiment the valve element is configured such that it switches back from said open state into said closed state if the pressure in said medium drops below a second threshold. This can be achieved by choosing a pre-stress, which is high enough, to bring the valve element out of the open state, for example. This results in very fast closing and response times, since the valve is closed as soon as the pressure of the medium in the channel drops below a certain threshold.

It is also possible to provide a valve element with a hysteresis, in order to provide a first threshold for opening the valve element and a second threshold for closing the valve element. The second threshold can especially be lower than the first threshold. It is also possible to design the open state as a meta-stable state.

It is preferred when the valve element has a convex shape in its closed position and a concave shape in its open position. In this way, it is possible to easily produce a valve element, which provides the necessary amount of flexibility. The embodiment is especially suitable to produce a meta-stable state, which would be the concave shape in this case.

It is further preferred when the medium is a gas or a fluid, in particular a medicament. In particular gases and fluids transmit the pressure efficiently, yielding a uniform pressure in the channel. In this way, pressure can be applied to the gas or fluid in a distance place, for example in a reservoir, while at the same time the pressure rises at the valve.

According to a last embodiment the medical device is a drug delivery device, a dispense interface for a drug delivery device or a needle hub. The use of a medical device according to the invention is especially useful for medical devices like these, since an effective sealing, a cost-saving production, an improved pressure control and an improved biocompatibility are desirable features for drug delivery devices and the like.

These as well as other advantages of various aspects of the present invention will become apparent to those of ordinary skill in the art by reading the following detailed description, with appropriate reference to the accompanying drawings.

### BRIEF DESCRIPTION OF THE FIGURES

FIG. 1 illustrates a perspective view of a delivery device with an end cap of the device removed;

FIG. 2 illustrates a perspective view of the delivery device distal end showing the cartridge;

FIG. 3 illustrates a perspective view of the delivery device illustrated in FIG. 1 or 2 with one cartridge retainer in an open position;

FIG. 4 illustrates a dispense interface and a dose dispenser that may be removably mounted on a distal end of the delivery device illustrated in FIG. 1;

FIG. 5 illustrates the dispense interface and the dose dispenser illustrated in FIG. 4 mounted on a distal end of the delivery device illustrated in FIG. 1;

FIG. 6 illustrates one arrangement of a needle assembly that may be mounted on a distal end of the delivery device;

FIG. 7 illustrates a perspective view of the dispense interface illustrated in FIG. 4;

FIG. 8 illustrates another perspective view of the dispense interface illustrated in FIG. 4;

FIG. 9 illustrates a cross-sectional view of the dispense interface illustrated in FIG. 4;

FIG. 10 illustrates an exploded view of the dispense interface illustrated in FIG. 4;

FIG. 11 illustrates a cross-sectional view of the dispense interface and needle assembly mounted onto a drug delivery device, such as the device illustrated in FIG. 1;

FIG. 12a illustrates an exemplary embodiment of a valve element and a channel according to the invention with the valve element in a closed state in a perspective view;

FIG. 12b illustrates the exemplary embodiment from FIG. 12a in a side view;

FIG. 13a illustrate an exemplary embodiment of a valve element and a channel according to the invention with the valve element in a closed state in a cross sectional view;

FIG. 13b illustrate the exemplary embodiment from FIG. 13a with the valve element in an open state in a cross sectional view;

FIG. 14a illustrates another exemplary embodiment of a valve element and a channel according to the invention with the valve element in a closed state in a perspective view;

FIG. 14b illustrates the exemplary embodiment from FIG. 14a in a side view;

FIG. 15 illustrates an exemplary embodiment of a valve element, a channel and a part of a medical device according to the invention in a cross sectional view.

### DETAILED DESCRIPTION

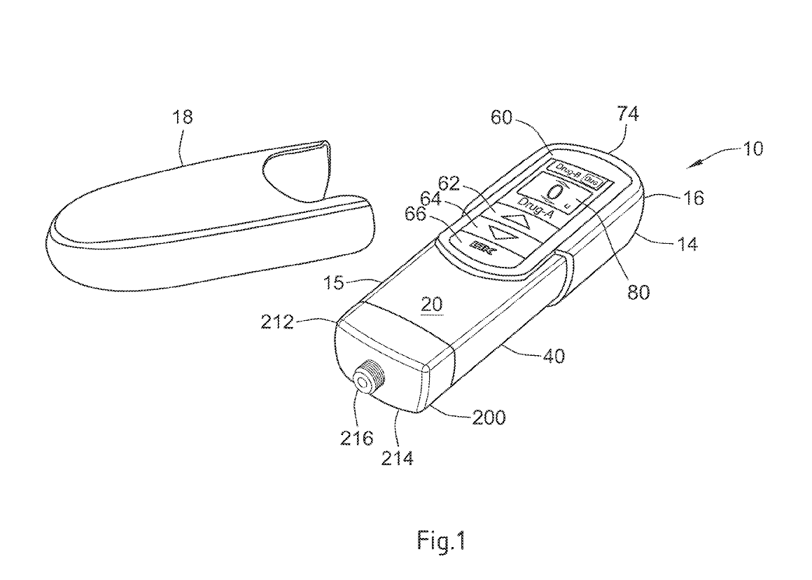

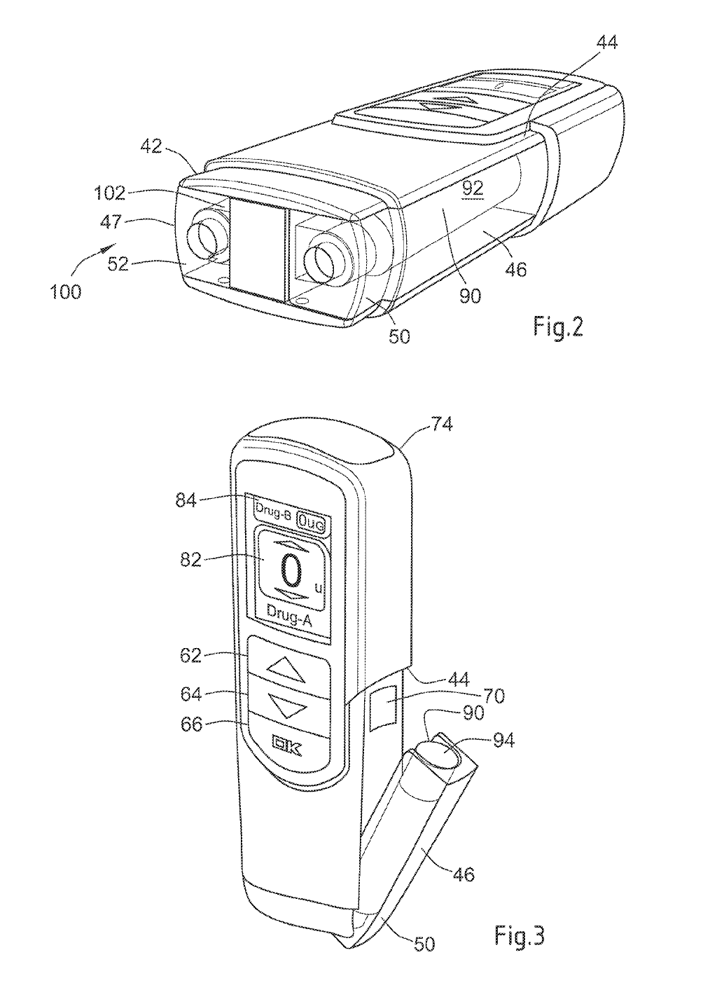

The drug delivery device illustrated in FIG. 1 comprises a main body 14 that extends from a proximal end 16 to a distal end 15. At the distal end 15, a removable end cap or cover 18 is provided. This end cap 18 and the distal end 15 of the main body 14 work together to provide a snap fit or form fit connection so that once the cover 18 is slid onto the distal end 15 of the main body 14, this frictional fit between the cap and the main body outer surface 20 prevents the cover from inadvertently falling off the main body.

The main body 14 contains a micro-processor control unit, an electro-mechanical drive train, and at least two medicament reservoirs. When the end cap or cover 18 is removed from the device 10 (as illustrated in FIG. 1), a dispense interface 200 is mounted to the distal end 15 of the main body 14, and a dose dispenser (e.g., a needle assembly) is attached to the interface. The drug delivery device 10 can be used to administer a computed dose of a second medicament (secondary drug compound) and a variable dose of a first medicament (primary drug compound) through a single needle assembly, such as a double ended needle assembly.

The drive train may exert a pressure on the bung of each cartridge, respectively, in order to expel the doses of the first and second medicaments. For example, a piston rod may push the bung of a cartridge forward a pre-determined amount for a single dose of medicament. When the cartridge is empty, the piston rod is retracted completely inside the main body 14, so that the empty cartridge can be removed and a new cartridge can be inserted.

A control panel region 60 is provided near the proximal end of the main body 14. Preferably, this control panel region 60 comprises a digital display 80 along with a plurality of human interface elements that can be manipulated by a user to set and inject a combined dose. In this arrangement, the control panel region comprises a first dose setting button 62, a second dose setting button 64 and a third button 66 designated with the symbol “OK.” In addition, along the most proximal end of the main body, an injection button 74 is also provided (not visible in the perspective view of FIG. 1).

The cartridge holder 40 can be removably attached to the main body 14 and may contain at least two cartridge retainers 50 and 52. Each retainer is configured so as to contain one medicament reservoir, such as a glass cartridge. Preferably, each cartridge contains a different medicament.

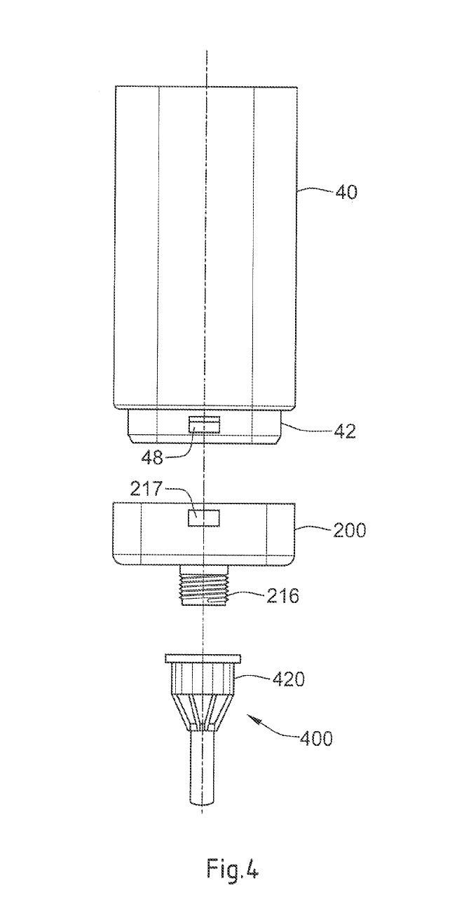

In addition, at the distal end of the cartridge holder 40, the drug delivery device illustrated in FIG. 1 includes a dispense interface 200. As will be described in relation to FIG. 4, in one arrangement, this dispense interface 200 includes a main outer body 212 that is removably attached to a distal end 42 of the cartridge housing 40. As can be seen in FIG. 1, a distal end 214 of the dispense interface 200 preferably comprises a needle hub 216. This needle hub 216 may be configured so as to allow a dose dispenser, such as a conventional pen type injection needle assembly, to be removably mounted to the drug delivery device 10.

Once the device is turned on, the digital display 80 shown in FIG. 1 illuminates and provides the user certain device information, preferably information relating to the medicaments contained within the cartridge holder 40. For example, the user is provided with certain information relating to both the primary medicament (Drug A) and the secondary medicament (Drug B).

As shown in FIG. 3, the first and a second cartridge retainers 50, 52 comprise hinged cartridge retainers. These hinged retainers allow user access to the cartridges. FIG. 3 illustrates a perspective view of the cartridge holder 40 illustrated in FIG. 1 with the first hinged cartridge retainer 50 in an open position. FIG. 3 illustrates how a user might access the first cartridge 90 by opening up the first retainer 50 and thereby having access to the first cartridge 90.

As mentioned above when discussing FIG. 1, a dispense interface 200 is coupled to the distal end of the cartridge holder 40. FIG. 4 illustrates a flat view of the dispense interface 200 unconnected to the distal end of the cartridge holder 40. A dose dispenser or needle assembly that may be used with the interface 200 is also illustrated and is provided in a protective outer cap 420.

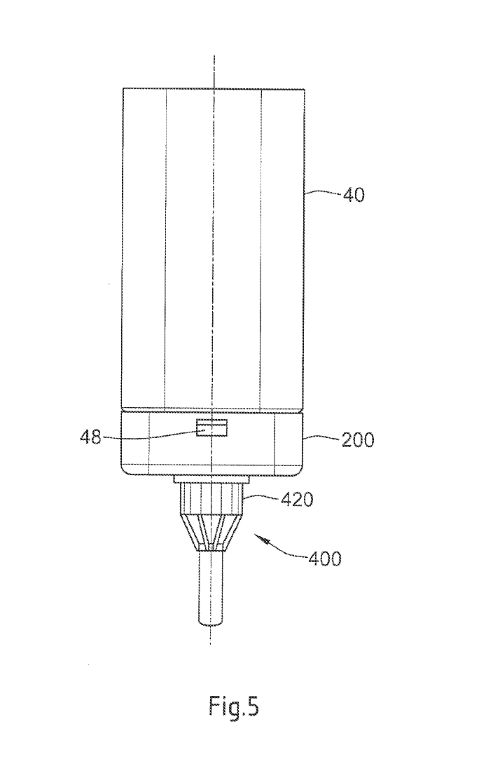

In FIG. 5, the dispense interface 200 illustrated in FIG. 4 is shown coupled to the cartridge holder 40. The axial attachment means between the dispense interface 200 and the cartridge holder 40 can be any known axial attachment means to those skilled in the art, including snap locks, snap fits, snap rings, keyed slots, and combinations of such connections. The connection or attachment between the dispense interface and the cartridge holder may also contain additional features (not shown), such as connectors, stops, splines, ribs, grooves, pips, clips and the like design features, that ensure that specific hubs are attachable only to matching drug delivery devices. Such additional features would prevent the insertion of a non-appropriate secondary cartridge to a non-matching injection device.

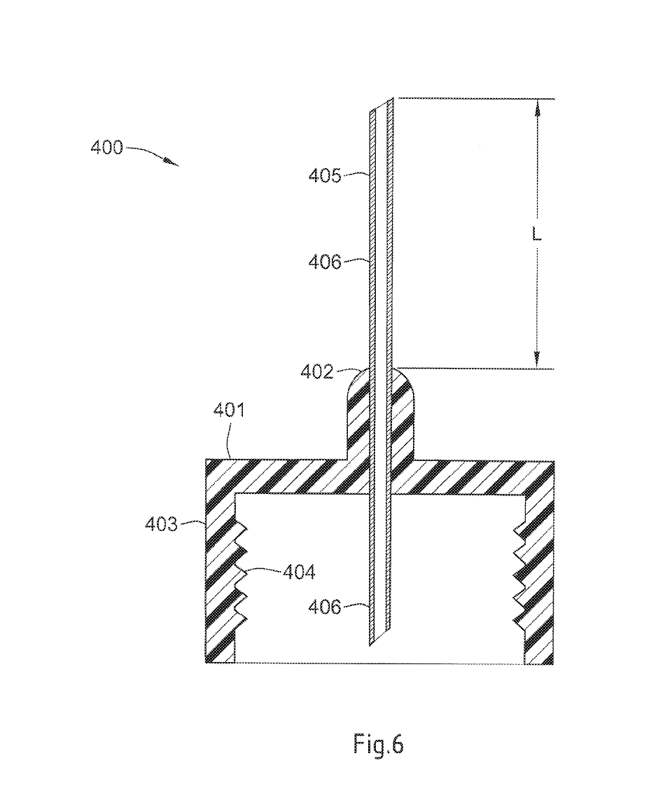

FIG. 5 also illustrates the needle assembly 400 and protective cover 420 coupled to the distal end of the dispense interface 200 that may be screwed onto the needle hub of the interface 200. FIG. 6 illustrates a cross sectional view of the double ended needle assembly 402 mounted on the dispense interface 200 in FIG. 5.

The needle assembly 400 illustrated in FIG. 6 comprises a double ended needle 406 and a hub 401. The double ended needle or cannula 406 is fixedly mounted in a needle hub 401. This needle hub 401 comprises a circular disk shaped element which has along its periphery a circumferential depending sleeve 403. Along an inner wall of this hub member 401, a thread 404 is provided. This thread 404 allows the needle hub 401 to be screwed onto the dispense interface 200 which, in one preferred arrangement, is provided with a corresponding outer thread along a distal hub. At a center portion of the hub element 401 there is provided a protrusion 402. This protrusion 402 projects from the hub in an opposite direction of the sleeve member. A double ended needle 406 is mounted centrally through the protrusion 402 and the needle hub 401. This double ended needle 406 is mounted such that a first or distal piercing end 405 of the double ended needle forms an injecting part for piercing an injection site (e.g., the skin of a user).

Similarly, a second or proximal piercing end 406 of the needle assembly 400 protrudes from an opposite side of the circular disc so that it is concentrically surrounded by the sleeve 403. In one needle assembly arrangement, the second or proximal piercing end 406 may be shorter than the sleeve 403 so that this sleeve to some extent protects the pointed end of the back sleeve. The needle cover cap 420 illustrated in FIGS. 4 and 5 provides a form fit around the outer surface 403 of the hub 401.

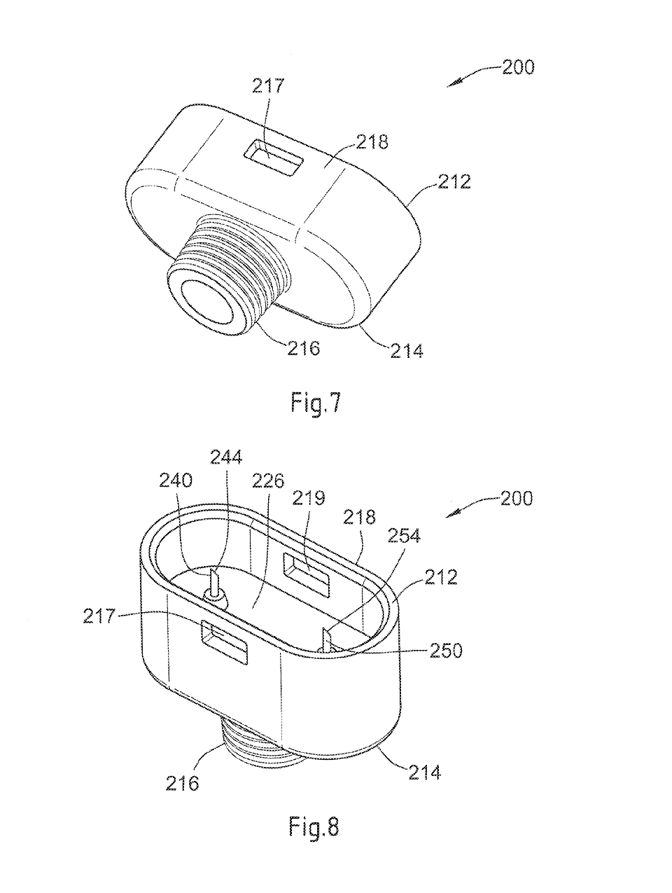

Referring now to FIGS. 4 to 11, one preferred arrangement of this interface 200 will now be discussed. In this one preferred arrangement, this interface 200 comprises:

a. a main outer body 210,

b. an first inner body 220,

c. a second inner body 230,

d. a first piercing needle 240,

e. a second piercing needle 250,

f. a valve seal 260, and

g. a septum 270.

The main outer body 210 comprises a main body proximal end 212 and a main body distal end 214. At the proximal end 212 of the outer body 210, a connecting member is configured so as to allow the dispense interface 200 to be attached to the distal end of the cartridge holder 40. Preferably, the connecting member is configured so as to allow the dispense interface 200 to be removably connected the cartridge holder 40. In one preferred interface arrangement, the proximal end of the interface 200 is configured with an upwardly extending wall 218 having at least one recess. For example, as may be seen from FIG. 8, the upwardly extending wall 218 comprises at least a first recess 217 and a second recess 219.

Preferably, the first and the second recesses 217, 219 are positioned within this main outer body wall so as to cooperate with an outwardly protruding member located near the distal end of the cartridge housing 40 of the drug delivery device 10. For example, this outwardly protruding member 48 of the cartridge housing may be seen in FIGS. 4 and 5. A second similar protruding member is provided on the opposite side of the cartridge housing. As such, when the interface 200 is axially slid over the distal end of the cartridge housing 40, the outwardly protruding members will cooperate with the first and second recess 217, 219 to form an interference fit, form fit, or snap lock. Alternatively, and as those of skill in the art will recognize, any other similar connection mechanism that allows for the dispense interface and the cartridge housing 40 to be axially coupled could be used as well.

The main outer body 210 and the distal end of the cartridge holder 40 act to form an axially engaging snap lock or snap fit arrangement that could be axially slid onto the distal end of the cartridge housing. In one alternative arrangement, the dispense interface 200 may be provided with a coding feature so as to prevent inadvertent dispense interface cross use. That is, the inner body of the hub could be geometrically configured so as to prevent an inadvertent cross use of one or more dispense interfaces.

A mounting hub is provided at a distal end of the main outer body 210 of the dispense interface 200. Such a mounting hub can be configured to be releasably connected to a needle assembly. As just one example, this connecting means 216 may comprise an outer thread that engages an inner thread provided along an inner wall surface of a needle hub of a needle assembly, such as the needle assembly 400 illustrated in FIG. 6. Alternative releasable connectors may also be provided such as a snap lock, a snap lock released through threads, a bayonet lock, a form fit, or other similar connection arrangements.

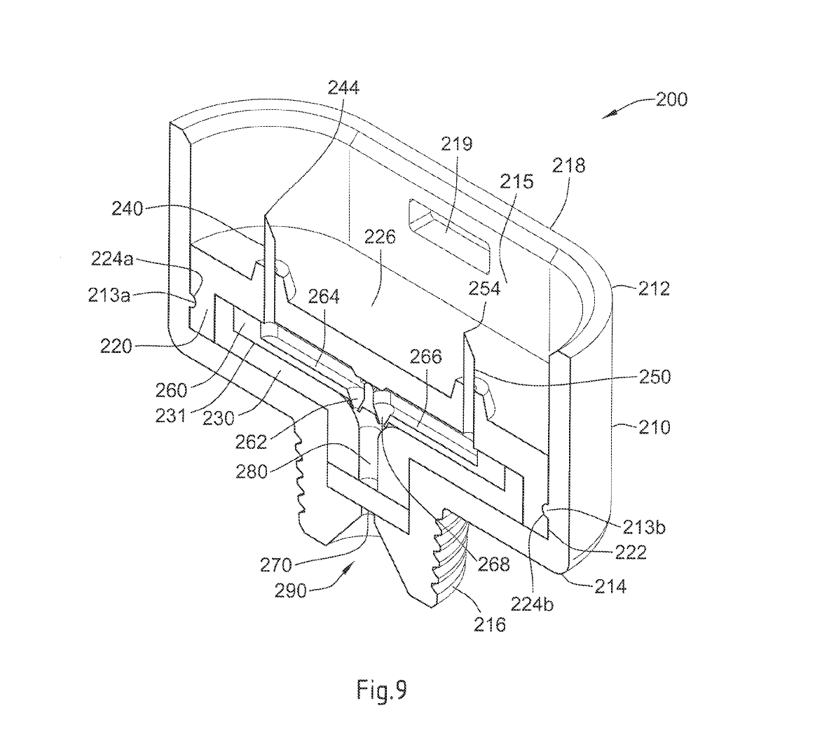

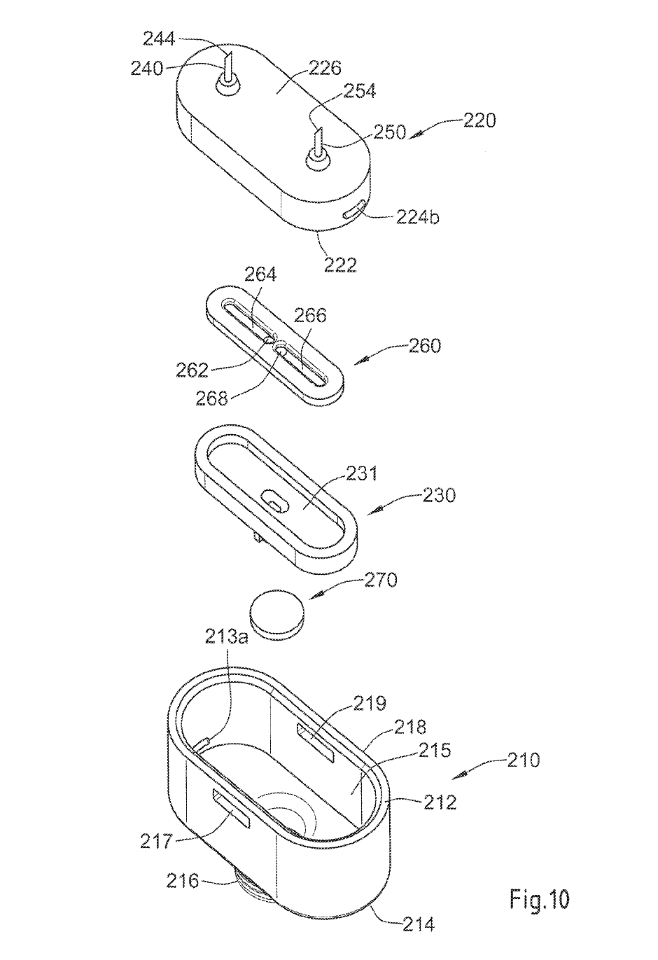

The dispense interface 200 further comprises a first inner body 220. Certain details of this inner body are illustrated in FIG. 8-11. Preferably, this first inner body 220 is coupled to an inner surface 215 of the extending wall 218 of the main outer body 210. More preferably, this first inner body 220 is coupled by way of a rib and groove form fit arrangement to an inner surface of the outer body 210. For example, as can be seen from FIG. 9, the extending wall 218 of the main outer body 210 is provided with a first rib 213a and a second rib 213b. This first rib 213a is also illustrated in FIG. 10. These ribs 213a and 213b are positioned along the inner surface 215 of the wall 218 of the outer body 210 and create a form fit or snap lock engagement with cooperating grooves 224a and 224b of the first inner body 220. In a preferred arrangement, these cooperating grooves 224a and 224b are provided along an outer surface 222 of the first inner body 220.

In addition, as can be seen in FIG. 8-10, a proximal surface 226 near the proximal end of the first inner body 220 may be configured with at least a first proximally positioned piercing needle 240 comprising a proximal piercing end portion 244. Similarly, the first inner body 220 is configured with a second proximally positioned piercing needle 250 comprising a proximally piercing end portion 254. Both the first and second needles 240, 250 are rigidly mounted on the proximal surface 226 of the first inner body 220.

Preferably, this dispense interface 200 further comprises a valve arrangement. Such a valve arrangement could be constructed so as to prevent cross contamination of the first and second medicaments contained in the first and second reservoirs, respectively. A preferred valve arrangement may also be configured so as to prevent back flow and cross contamination of the first and second medicaments.

In one preferred system, dispense interface 200 includes a valve arrangement in the form of a valve seal 260. Such a valve seal 260 may be provided within a cavity 231 defined by the second inner body 230, so as to form a holding chamber 280. Preferably, cavity 231 resides along an upper surface of the second inner body 230. This valve seal comprises an upper surface that defines both a first fluid groove 264 and second fluid groove 266. For example, FIG. 9 illustrates the position of the valve seal 260, seated between the first inner body 220 and the second inner body 230. During an injection step, this seal valve 260 helps to prevent the primary medicament in the first pathway from migrating to the secondary medicament in the second pathway, while also preventing the secondary medicament in the second pathway from migrating to the primary medicament in the first pathway. Preferably, this seal valve 260 comprises a first non-return valve 262 and a second non-return valve 268. As such, the first non-return valve 262 prevents fluid transferring along the first fluid pathway 264, for example a groove in the seal valve 260, from returning back into this pathway 264. Similarly, the second non-return valve 268 prevents fluid transferring along the second fluid pathway 266 from returning back into this pathway 266.

Together, the first and second grooves 264, 266 converge towards the non-return valves 262 and 268 respectively, to then provide for an output fluid path or a holding chamber 280. This holding chamber 280 is defined by an inner chamber defined by a distal end of the second inner body both the first and the second non return valves 262, 268 along with a pierceable septum 270. As illustrated, this pierceable septum 270 is positioned between a distal end portion of the second inner body 230 and an inner surface defined by the needle hub of the main outer body 210.

The holding chamber 280 terminates at an outlet port of the interface 200. This outlet port 290 is preferably centrally located in the needle hub of the interface 200 and assists in maintaining the pierceable seal 270 in a stationary position. As such, when a double ended needle assembly is attached to the needle hub of the interface (such as the double ended needle illustrated in FIG. 6), the output fluid path allows both medicaments to be in fluid communication with the attached needle assembly.

The hub interface 200 further comprises a second inner body 230. As can be seen from FIG. 9, this second inner body 230 has an upper surface that defines a recess, and the valve seal 260 is positioned within this recess. Therefore, when the interface 200 is assembled as shown in FIG. 9, the second inner body 230 will be positioned between a distal end of the outer body 210 and the first inner body 220. Together, second inner body 230 and the main outer body hold the septum 270 in place. The distal end of the inner body 230 may also form a cavity or holding chamber that can be configured to be fluid communication with both the first groove 264 and the second groove 266 of the valve seal.

Axially sliding the main outer body 210 over the distal end of the drug delivery device attaches the dispense interface 200 to the multi-use device. In this manner, a fluid communication may be created between the first needle 240 and the second needle 250 with the primary medicament of the first cartridge and the secondary medicament of the second cartridge, respectively.

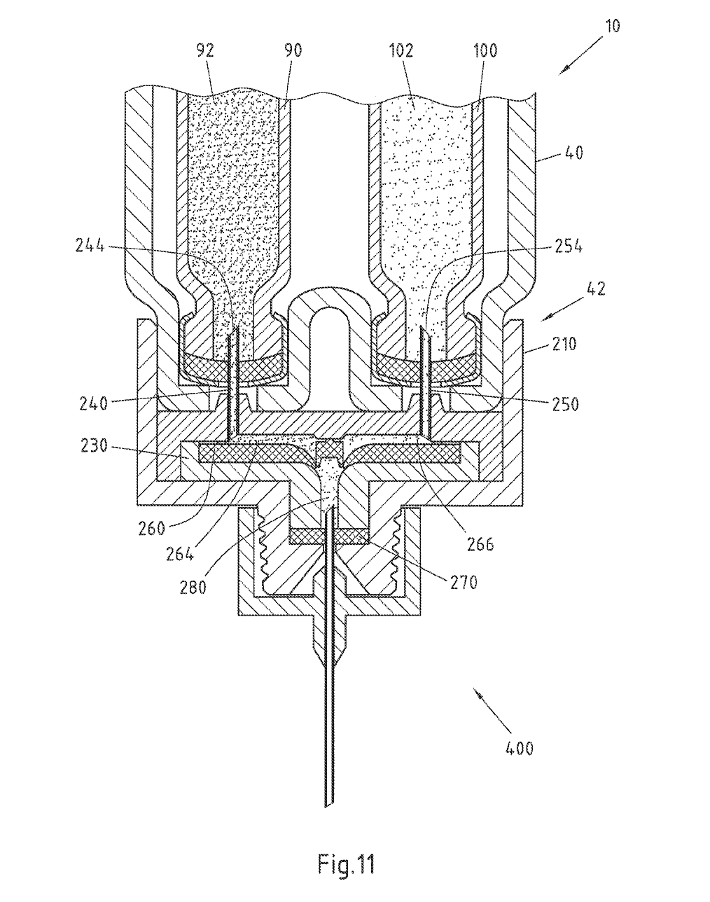

FIG. 11 illustrates the dispense interface 200 after it has been mounted onto the distal end 42 of the cartridge holder 40 of the drug delivery device 10 illustrated in FIG. 1. A double ended needle 400 is also mounted to the distal end of this interface. The cartridge holder 40 is illustrated as having a first cartridge containing a first medicament and a second cartridge containing a second medicament.

When the interface 200 is first mounted over the distal end of the cartridge holder 40, the proximal piercing end 244 of the first piercing needle 240 pierces the septum of the first cartridge 90 and thereby resides in fluid communication with the primary medicament 92 of the first cartridge 90. A distal end of the first piercing needle 240 will also be in fluid communication with a first fluid path groove 264 defined by the valve seal 260.

Similarly, the proximal piercing end 254 of the second piercing needle 250 pierces the septum of the second cartridge 100 and thereby resides in fluid communication with the secondary medicament 102 of the second cartridge 100. A distal end of this second piercing needle 250 will also be in fluid communication with a second fluid path groove 266 defined by the valve seal 260.

FIG. 11 illustrates a preferred arrangement of such a dispense interface 200 that is coupled to a distal end 15 of the main body 14 of drug delivery device 10. Preferably, such a dispense interface 200 is removably coupled to the cartridge holder 40 of the drug delivery device 10.

As illustrated in FIG. 11, the dispense interface 200 is coupled to the distal end of a cartridge housing 40. This cartridge holder 40 is illustrated as containing the first cartridge 90 containing the primary medicament 92 and the second cartridge 100 containing the secondary medicament 102. Once coupled to the cartridge housing 40, the dispense interface 200 essentially provides a mechanism for providing a fluid communication path from the first and second cartridges 90, 100 to the common holding chamber 280. This holding chamber 280 is illustrated as being in fluid communication with a dose dispenser. Here, as illustrated, this dose dispenser comprises the double ended needle assembly 400. As illustrated, the proximal end of the double ended needle assembly is in fluid communication with the chamber 280.

In one preferred arrangement, the dispense interface is configured so that it attaches to the main body in only one orientation, that is it is fitted only one way round. As such as illustrated in FIG. 11, once the dispense interface 200 is attached to the cartridge holder 40, the primary needle 240 can only be used for fluid communication with the primary medicament 92 of the first cartridge 90 and the interface 200 would be prevented from being reattached to the holder 40 so that the primary needle 240 could now be used for fluid communication with the secondary medicament 102 of the second cartridge 100. Such a one way around connecting mechanism may help to reduce potential cross contamination between the two medicaments 92 and 102.

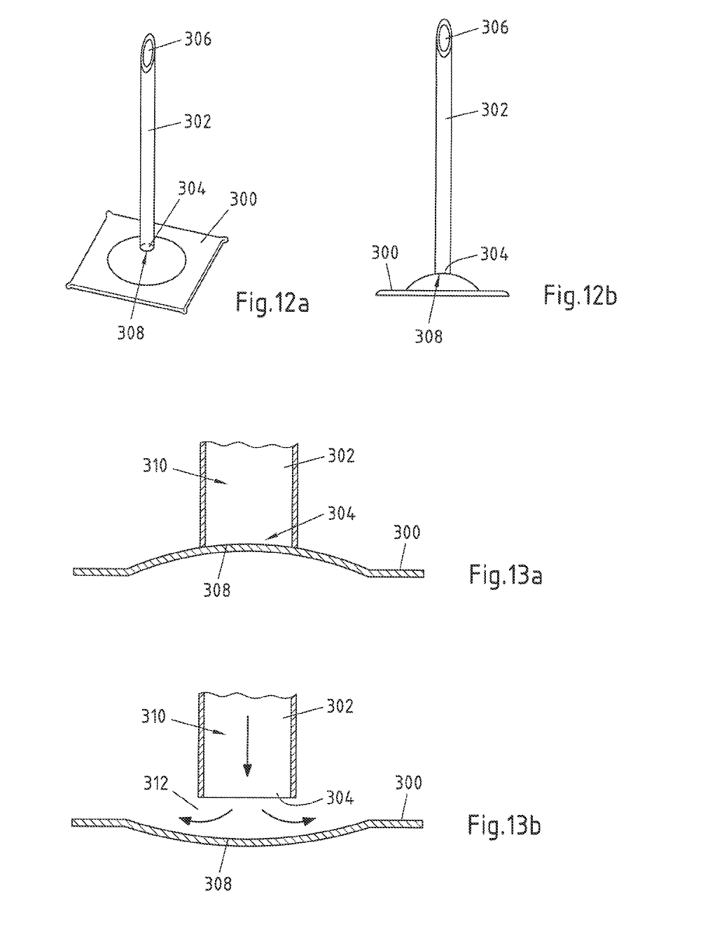

FIG. 12a illustrates an exemplary embodiment of a valve element 300 and a channel 302 according to the invention with the valve element 300 in a closed state in a perspective view. The valve element 300 substantially comprises a rectangular metal sheet, which is raised by a convex curvature in its center. The valve element 300 is preferably fixed at one or more of the edges of the rectangle. The valve element 300 can of course be of any other geometric form or material. The valve element further comprises a sealing area 308, which is in contact with a channel 302. The channel 302 has two openings 304 and 306. The opening 304 is sealed by the sealing area 308 of valve element 300.

In this case the channel 302 is a needle, which is preferably made of metal. In particular, the channel may comprise the needle 240 and/or 250 illustrated in FIG. 9.

FIG. 12b illustrates the exemplary embodiment from FIG. 12a in a side view. It can be seen that the sealing area 308 of the valve element 300 seals the opening 304 of the channel 302. It is important to notice that the valve element 300 and the channel 302 and not connected with each other, but only in contact with each other.

FIG. 13a illustrates an exemplary embodiment of a valve element 300 and a channel 302 according to the invention with the valve element 300 in a closed state in a cross sectional view. The illustrated embodiment is similar to the one illustrated in FIG. 12. The channel 302 guides a medium 310, in particular a fluid, especially a medicament or a drug, though other mediums are possible as well. The medium 310 cannot exit the channel 302 through the opening 304 since it is sealed by the sealing area 308 of the valve element 300. The fluid can have a pressure, which is too low to deform the flexible valve element 300, for example a substantially ambient pressure.

FIG. 13b illustrates the exemplary embodiment from FIG. 13a with the valve element 300 in an open state in a cross sectional view. When the pressure of the medium 310 in the channel 302 is increased, the medium can deform the valve element from a convex curvature into a concave curvature. This may be a meta-stable state. The concave curvature of the valve element 300 produces a circumferential opening or slit 312 between the sealing area 308 and the channel 302, thorough which the medium 310 can exit the channel 302, as shown by the arrows in FIG. 13b.

If the pressure of the medium 310 in the channel 302 is decreased again, the force against the valve element 300, in particular against the sealing area 308 is reduced, the valve element 300 may either remain in the open state or not, depending on whether the valve element 300 is pre-stressed such that this tension can bring the valve element 300 from the open state back into the closed, convex state. In case the valve element 300 remains in the open state, it may be closed again when a pressure outside the channel 302 increases that would press the fluid back into the channel 302.

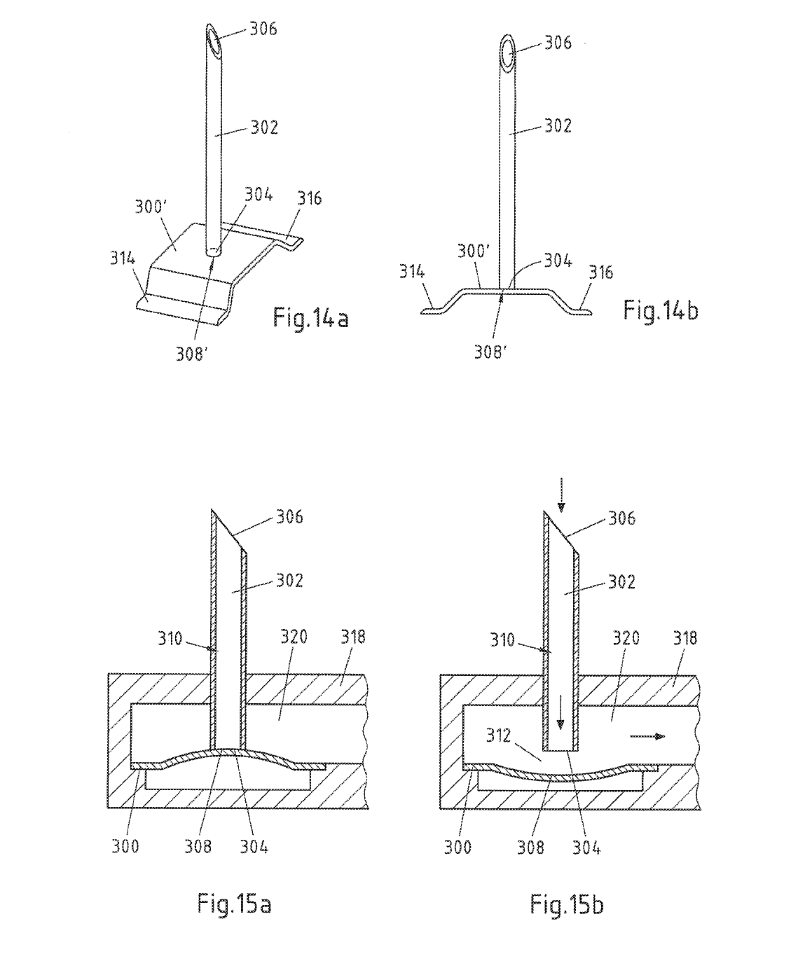

FIG. 14a and FIG. 14b illustrate another exemplary embodiment of a valve element 300′ and a channel 302 according to the invention with the valve element 300′ in a closed state in a perspective view and in a side view, respectively. In contrast to the embodiment illustrated in FIG. 12 the valve element 300′ shown in FIG. 14a is designed as a spring element. The sealing area 308′ is raised compared to the edge areas 314 and 316 of the valve element 300′. The edge areas 314, 316 are preferentially fixed. In this case due to the stable geometric form of the valve element 300′, it is not possible to bring the valve element 300′ in a meta-stable state, which is comparable to the state with the concave shape of the valve element 300. Thus there is a constant force acting against the pressure of the medium 312, even if the valve element 300′ is an open state. Thus a faster closing is possible while the pressure of the medium 312 decreases. The material of the valve element 300′ may be flexible, so that a pressure of a fluid in the channel 302 may slightly bend the sealing area 308′ downward. Thus, the fluid can be pressed out of the channel 302 through a slit or opening between the channel 302 and the sealing area 308′. When the pressure of the fluid in the channel 302 is reduced, the slit or opening will gradually become smaller and eventually close again, for example when the pressure of the fluid inside the channel 302 is substantially the same as the pressure outside the channel 302. The degree of flexibility of the valve element 300′ may be determined by the material of the valve element 300′ as well as the geometric properties, for example the thickness of the valve element 300′, the height and angle of the flanges, the size of the sealing area 308′, and/or the like.

FIG. 15 illustrates an exemplary embodiment of a valve element 300, a channel 302 and a part 318 of a medical device according to the invention in a cross sectional view. The valve element 300 and the channel 302 are similar to the ones illustrated in FIG. 12. A medium 312 might be guided from a reservoir, for example, through the channel 302 in the direction of the arrows. In the closed state of the valve element 300 the medium 312 is stopped at the sealing area 308 of the valve element 300. If the pressure is big enough the valve element 300 can switch to the open state, which state is illustrated by FIG. 15b. Now the medium 312 can pass the valve element 300 through a slit or opening between the channel 302 and the valve element 300 and enter the channel 320. This channel can lead to another needle or a cannula in order to guide the fluid to an injection site, for example. The part 318 can specially be an arm of a y-channel, which is implemented in a medical device, as shown in FIGS. 9 and 11. The succeeding channel 320 can in particular be the fluid groove 264 or 266 and the medical device especially a dispense interface 200 or a drug delivery device 10.

The valves 262 and 268 as shown in FIGS. 9 and 10 would be dispensable if a valve element according to the invention is used. It is also possible to implement valve elements 300, 300′ in the location of the valves 262 and 268.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39),

wherein the group -Lys6-NH2 may be bound to the C-terminus of the Exendin-4 derivative;
or an Exendin-4 derivative of the sequence

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(O2)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(O)14, Asp28] Exendin-4(1-39)-Lys6-NH2,

### des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

### H-Asn-(Glu)5-des Pro36, Pro37, Pro38 [Met(O)14, Asp28] Exendin-4(1-39)-NH2,

### des Pro36, Pro37, Pro38 [Met(O)14, Asp28] Exendin-4(1-39)-(Lys)6-NH2,

### H-(Lys)6-des Pro36, Pro37, Pro38 [Met(O)14, Asp28] Exendin-4(1-39)-(Lys)6-NH2,

### H-Asn-(Glu)5 des Pro36, Pro37, Pro38 [Met(O)14, Asp28] Exendin-4(1-39)-(Lys)6-NH2,

### H-Lys6-des Pro36 [Met(O)14, Trp(O2)25, Asp28] Exendin-4(1-39)-Lys6-NH2,

### H-des Asp28 Pro36, Pro37, Pro38 [Met(O)14, Trp(O2)25] Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Trp(O2)25, Asp28] Exendin-4(1-39)-NH2,

### des Pro36, Pro37, Pro38 [Met(O)14, Trp(O2)25, Asp28] Exendin-4(1-39)-(Lys)6-NH2,

### H-(Lys)6-des Pro36, Pro37, Pro38 [Met(O)14, Trp(O2)25, Asp28] Exendin-4(S1-39)-(Lys)6-NH2,

### H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystallizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

1-14. (canceled)

15. A medical device, comprising:
a channel comprising at least two openings,
a valve element comprising at least one sealing area,
wherein said valve element is at least in part flexible,
wherein said valve element is configured to have a closed state, in which said sealing area of said valve element seals one of said openings of said channel,
wherein said valve element is further configured to have an open state, such that said valve element can be brought into said open state by the pressure of a medium inside said channel, and said medium can exit said channel between said valve element and said channel and
wherein said valve element is at least partially made of metal.

16. The medical device according to claim 15, wherein the sealing area of said valve element, when in said closed state, seals said opening of said channel by direct contact with said channel.

17. The medical device according to claim 15, wherein said sealing area of said valve element by the pressure of said medium inside said channel can be bent in the axial direction of said channel and away from said channel.

18. The medical device according to claim 15, wherein said valve element is at least partially made of titanium or steel.

19. The medical device according to claim 15, wherein said channel is at least in part made of metal, in particular titanium or steel.

20. The medical device according to claim 15, wherein said valve element is substantially flat.

21. The medical device according to claim 15, wherein said valve element, in its closed state, is curved towards said channel.

22. The medical device according to claim 15, wherein said valve element is pre-stressed, such that said valve element returns into said closed state if the pressure in said medium drops below a threshold.

23. The medical device according to claim 15, wherein said valve element shows hysteresis during its opening and closing.

24. The medical device according to claim 23, wherein said valve element is configured such that it substantially remains in said open state, even if the pressure in said medium drops.

25. The medical device according to claim 23, wherein said valve element is configured such that it switches back from said open state into said closed state if the pressure in said medium drops below a second threshold.

26. The medical device according to claim 15, wherein said valve element has a convex shape in its closed position and a concave shape in its open position.

27. The medical device according to claim 15, wherein said medium is a gas or a fluid, in particular a medicament.

28. The medical device according to claim 15, wherein said medical device is a drug delivery device, a dispense interface for a drug delivery device or a needle hub.
